(19) United States
(12) Patent Application Publication
(10) Pub. No.: US 2006/0024270 A1
(43) Pub. Date: Feb. 2, 2006

(54) Compositions and methods for preventing or treating encephalitis with interferon

(75) Inventor: James J. Rahal, New York, NY (US)

Correspondence Address: KALOW & SPRINGUT LLP, 488 MADISON AVENUE, 19TH FLOOR, NEW YORK, NY, 10022, US

(73) Assignee: The New York Hospital Medical Center Of Queens, Flushing, NY (US)

(21) Appl. No.: 11/231,433

(22) Filed: Sep. 20, 2005

Related U.S. Application Data
(63) Continuation-in-part of application No. 09/935,966, filed on Aug. 23, 2001, now Pat. No. 6,946,125.
(60) Provisional application No. 60/227,422, filed on Aug. 23, 2000.

(51) Int. Cl. A61K 38/21

(52) U.S. Cl. 424/85.4; 424/85.7

(57) ABSTRACT

Compositions and methods are provided for preventing or treating a human suffering from a meningitis, encephalitis, meningo-encephalitis or encephalomyelitis caused by an arbovirus, comprising parenterally administering to the human an effective amount of interferon.

This application is a continuation-in-part application of Ser. No. 09/935,966 filed Aug. 23, 2001, entitled “Methods of Preventing or Treating West Nile Virus and Other Infections” which claims the benefit of the filing date of Provisional Application Ser. No. 60/227,422, filed Aug. 23, 2000, entitled “Methods for Treating West Nile Virus” these entire disclosure are hereby incorporated by reference into the present disclosure.

### BACKGROUND

Encephalitis or inflammation of the brain is a potentially life-threatening condition occurring in patients of all ages resulting in hospitalization and often death. Most causes of encephalitis are unknown. However, of the known causes of encephalitis, viral encephalitis tends to be the most common with arboviruses being implicated as the cause of many viral cases of encephalitis. Recently, there has been great interest in a particular arbovirus subgroup called flaviviruses, which include West Nile virus, St. Louis encephalitis virus and others.

West Nile virus (WNV) an arthropod-borne flavivirus, has emerged as a deadly health threat to not only humans, but also to other animal species such as horses and birds. New York was the first area in North American to report cases of WNV infections. WNV infection in humans has been found previously only in Africa, the Middle East and Eastern Europe. The virus is transmitted to humans and several animal species by mosquitoes, which acquire the virus by feeding on infected birds. It is not known how WNV was transmitted into the United States, however, scientists believe that it may have been transmitted through immigration or importation of an infected human, bird or mosquito from an endemic area.

WNV, as well as other arbovirus, remain a continued threat to public health. Epidemiologic and virologic studies indicate that live WNV persists in mosquito and bird populations. Mosquito control measures were implemented in New York, New Jersey and Connecticut, yet new cases of WNV are being diagnosed. Certain populations are still being exposed to West Nile virus. For example, WNV sero-prevalence studies in Queens, New York indicate that 2.6% of the population, age 5 or older, had evidence of prior infection. Thus, a large portion of the population in Queens, and probably a larger portion in surrounding areas, remains susceptible to WNV.

Among humans infected with WNV, approximately one in every 150 will have evidence of meningitis (headache, stiff neck) or encephalitis (change of mental status, peripheral neurologic abnormalities, muscle weakness). Though most humans with meningitis or encephalitis in New York were over age 50, older age was not associated with meningoencephalitis among 393 cases reported in the 1996 Romanian outbreak. However, almost all fatalities have occurred among humans over the age of 50. Underlying preexisting medical conditions were present in several fatal cases in New York, but were not risk factors for meningoencephalitis in Romania. The fatality rate among patients with central nervous system infection was 5% in Romania and 11% in New York. Fatalities have been due to prolonged central nervous system dysfunction requiring ventilatory support and leading to secondary complications. Prolonged neurologic symptoms have occurred in survivors of encephalitis.

Prior to the New York outbreak, fatal infection in birds (i.e. crows) was unusual. The New York strain of WNV is most closely related to an isolated virus from a dead goose in Israel where increased pathogenicity of WNV for birds was also noted.

The diagnosis of acute WNV infection in humans is established by the presence of IgM antibody in serum or cerebrospinal fluid, a four-fold increase in antibody by ELISA or neutralization, or identification of WNV RNA in brain tissue by polymerase chain reaction, or viral isolation. Occasionally, viremia (virus is in blood) occurs, but isolation of WNV from blood has been uncommon.

Until the present invention, prevention or treatment of WNV infection was merely supportive (e.g., anti-pyretics are given to keep fever down, fluids, antibiotics for secondary bacterial infection, respiratory support as necessary, etc). One author in an abstract described activity of ribavirin against WNV infection in mice.

Ribavirin and interferon alpha-2b have been used to treat hepatitis C virus infections which is a member of the genus Flavivirus. However, hepatitis C virus infections are localized viral infections of the liver that can result in diseases such as cirrhosis, decompensated liver disease or hepatocellular carcinoma. These localized diseases affect primarily the liver and not the central nervous system as seen in arbovirus infections.

Another potentially life-threatening arborvirus is St. Louis encephalitis (SLE) virus. An outbreak of St. Louis meningoencephalitis due to SLE virus occurred in northeastern Louisiana during the summer of 2001. Thirteen patients were admitted to the same hospital during the first 3 weeks of August. Three became quadriplegic and required ventilatory assistance during the first week of hospitalization. Until the present invention, treatment for SLE was merely supportive care.

Based on the foregoing, there is a need for effective compositions and methods for preventing or treating encephalitis caused by arboviruses (e.g., West Nile virus, SLE, Japanese encephalitis virus, etc.). There is also a need for effective combination therapy to prevent or treat encephalitis caused by arbovirus.

### SUMMARY OF THE INVENTION

The present invention provides compositions and methods for preventing or treating humans suffering from meningitis, encephalitis, meningo-encephalitis or encephalomyelitis caused by an arbovirus, comprising parenterally administering to the human an effective amount of interferon.

In one embodiment, a method is provided for treating a human suffering from a meningitis, encephalitis, meningo-encephalitis or encephalomyelitis caused by an arbovirus, comprising parenterally administering to the human an effective amount of interferon.

In another embodiment, a method is provided for preventing or treating a human suffering from a meningitis, encephalitis, meningo-encephalitis or encephalomyelitis caused by a West Nile virus, comprising parenterally administering to the human an effective amount of interferon.

In one exemplary embodiment, a method is provided for treating a human suffering from a meningitis, encephalitis, meningo-encephalitis or encephalomyelitis caused by St. Louis encephalitis virus, comprising parenterally administering to the human an effective amount of interferon.

In another exemplary embodiment, a method is provided for treating a human suffering from a meningitis, encephalitis, meningo-encephalitis or encephalomyelitis caused by a West Nile virus, comprising intravenously and/or subcutaneously administering to the human an effective amount of interferon-alpha.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE FIGURES

Preferred embodiments of the invention have been chosen for purposes of illustration and description, but are not intended in any way to restrict the scope of the invention. The preferred embodiments of certain aspects of the invention are shown in the accompanying figures, wherein:

FIG. 1 illustrates effects of varying concentrations of interferon alpha-2b on WNV infected Vero cells. The vertical axis represents a colorimetric assay of cellular lactic dehydrogenase which is directly proportional to cell viability and proliferation. The horizontal axis represents increasing concentrations of alpha interferon added to infected and uninfected cells, as such concentrations increase, the viability of infected cells approaches that of uninfected cells illustrating the therapeutic action of interferon.

FIG. 2 illustrates effects of varying concentrations of ribavirin plus a constant subinhibitory concentration of interferon alpha 2b (0.37 units/ml) on WNV infected Vero cells. The vertical axis represents a colorimetric assay of cellular lactic dehydrogenase, which is directly proportional to cell viability and proliferation. The horizontal axis represents increasing concentrations of ribavirin added to infected and uninfected cells. As such concentrations approach 400AM, the viability of infected cells approaches that of uninfected cells, illustrating a therapeutic action of ribavirin plus interferon alpha-2b. However, as the ribavirin concentration exceeds 400 μM, the viability of infected cells declines, consistent with toxicity due to ribavirin.

FIG. 3 is a graphic illustration of combined neurologic function scores, over time, for 15 patients with St. Louis meningoencephalitis treated with interferon alpha 2b and for 17 untreated patients. The numbers in parentheses denote the number of patients seen for follow-up at each weekly interval. The neurologic function scores of treated patients were statistically greater than those of untreated patients three weeks after onset of observation and therapy.

### DETAILED DESCRIPTION OF THE INVENTION

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

In various embodiments, Applicant observed that interferon therapy is useful for preventing or treating meningitis, encephalitis, meningo-encephalitis or encephalomyelitis caused by an arbovirus. In various embodiments, Applicant observed that ribavirin in vitro was effective against arbovirus.

Arboviruses include, but are not limited to, flaviviruses, which include, but are not limited to, WNV encephalitis, St. Louis encephalitis virus, Murray Valley encephalitis virus, Japanese encephalitis virus, eastern equine encephalitis virus, western equine encephalitis virus, LaCrosse encephalitis virus, tick-borne encephalitis, Venezuelan equine encephalitis or Powassan virus encephalitis.

In various embodiments, interferon is used to prevent or treat meningitis, encephalitis, meningo-encephalitis or encephalomyelitis caused by an arbovirus.

The diagnosis of arbovirus infections, such as WNV, in humans can be established by the presence of arbovirus IgM antibody (e.g., WNV IgM antibody) in serum or cerebrospinal fluid, increases in arbovirus antibody detected by ELISA or arbovirus neutralizing antibody, identification of arbovirus RNA in tissue by polymerase chain reaction, and arbovirus viral isolation in fluids such as in cerebral spinal fluid and/or blood.

Some medical symptoms, syndromes, conditions or diseases associated with WNV encephalitis, St. Louis encephalitis virus, Murray Valley encephalitis virus, Japanese encephalitis virus, eastern equine encephalitis virus, western equine encephalitis virus, LaCrosse encephalitis virus, tick-borne encephalitis, Venezuelan equine encephalitis or Powassan virus encephalitis, include but are not limited to one or more of the following: infection, viremia, stiff neck, headache, fever, myalgia, change of mental status, peripheral neurologic abnormalities, muscle weakness, rash, meningitis, encephalitis, encephalomyelitis and/or meningoencephalitis.

Typically, fatalities result from encephalitis or meningo-encephalitis. As used herein, encephalitis is an art recognized term and includes inflammation of the brain. Meningitis includes inflammation of the meninges of the brain. Meningo-encephalitis is a combination of meningitis and encephalitis and includes inflammation of the spinal cord covering (meninges) and brain. Encephalomyelitis is a general term for inflammation of the brain and nerves in the spinal cord.

Some types of encephalitis associated with arbovirus infections include, but are not limited to, WNV encephalitis, St. Louis encephalitis virus, Murray Valley encephalitis virus, Japanese encephalitis virus, eastern equine encephalitis virus, western equine encephalitis virus, LaCrosse encephalitis virus, tick-borne encephalitis, Venezuelan equine encephalitis or Powassan virus encephalitis.

St. Louis encephalitis (SLE) virus, like WNV, is also caused by an arbovirus known as a flavivirus. It is carried by Culex mosquitoes (although other mosquitoes may also transmit it to humans). It takes its name from an epidemic in St. Louis, but outbreaks have occurred in wider geographic areas, especially in midwestern and southeastern states, and can occur in rural or urban areas. As of 2000, the highest numbers of total cases have been reported in Texas (970), Illinois (695), Ohio (440), Indiana (368), and Florida (379). Mild congestion in blood vessels of the brain with small areas of bleeding as well as mild infiltration of infection into the meninges has been reported. SLE infection accumulates in both gray and white matter, where the thalamus and midbrain are more likely to be affected.

Japanese encephalitis (JE) virus, like WNV, is also caused by an arbovirus known as a flavivirus and is transmitted to humans by infected mosquitoes. It is the most common viral encephalitis outside the US and occurs in rural areas in east, south, and southwest Asia, especially China and Korea. JE virus infection occurs mainly in children and is usually mild. If symptoms of encephalitis develop, it has a very high mortality rate. A formalin-inactivated vaccine prepared in mice is used widely in Japan, China, India, Korea, Taiwan and Thailand. This vaccine is currently available for human use in the United States, for individuals who might be traveling to endemic countries.

Murray Valley encephalitis (MVE) is endemic in New Guinea and in parts of Australia; and is related to SLE, WNV and JE viruses. Unapparent infections are common, and the small number of fatalities reported have mostly been in children.

Eastern equine encephalitis (EEE) virus typically begins its life cycle as a parasite of migratory wild birds and is transferred through Aedes albopictus mosquito bites to either horses or humans. The virus is found in Atlantic and Gulf coasts, in New England, and around the Great Lakes. EEE can cause congestion of blood vessels in the brain and widespread changes in nerve cells. Lesions can be found in white and gray matter. EEE can also affect all major parts of the brain. The virus can induce seizures, palsy, mental impairment, partial paralysis, and speech problems, which are usually more severe in children. About 182 cases have been confirmed since 1964. Mortality rates are about 30% to 80%. Children are more likely to survive but also to suffer complications afterward.

Western equine encephalitis (WEE) virus begins its life cycle as a parasite of migratory wild birds and is transferred through mosquito bites to either horses or humans. Snakes and rodents may also harbor the virus. WEE occurs throughout the country, but mostly in rural regions west of the Mississippi. WEE causes less inflammation and fewer nerve cell changes than the Eastern variant. The virus can induce seizures, palsy, mental impairment, partial paralysis, and speech problems, which are usually more severe in children. The mortality rate for WEE is 3% to 4%; 30% of survivors have complications afterward. Most severe complications are in children.

La Crosse encephalitis (a California serogroup virus), typically, is caused by an arbovirus known as bunyavirus. Its original hosts are small animals, such as chipmunks and squirrels. It is transmitted to humans by infected Aedes triseriatus mosquitoes. The La Crosse virus, originally isolated in La Crosse Wisconsin in 1965, is the most serious of the California encephalitis viruses. La Crosse encephalitis occurs most frequently in the north central US and along the eastern seaboard. Most cases have occurred in Ohio and Wisconsin. The virus affects mostly children and may cause emotional difficulties, learning problems, or seizures. Neurologic effects usually resolve after several years. La Crosse encephalitis causes on average about 113 cases a year and is usually very mild, though the number of cases is increasing and many cases may go undiagnosed or unreported. Mortality rates are less than 1%. La Crosse encephalitis is more common and severe in children under age 16.

Powassan (POW) virus is a flavivirus and currently the only well documented tick-borne transmitted arbovirus occurring in the United States and Canada. Recently a Powassan-like virus was isolated from the deer tick, Ixodes scapularis. Its relationship to POW and its ability to cause human disease has not been fully elucidated. POW's range in the United States is primarily in the upper tier States. In addition to isolations from man, the virus has been recovered from ticks (Ixodes marxi, I. cookei and Dermacentor andersoni) and from the tissues of a skunk (Spiligale putorius). It is a rare cause of acute viral encephalitis. POW virus was first isolated from the brain of a 5-year-old child who died in Ontario in 1958. Patients who recover may have residual neurological problems.

Tick-borne encephalitis (TBE) is caused by two closely related flaviviruses which are distinct biologically. The eastern subtype causes Russian spring-summer encephalitis (RSSE) and is transmitted by Ixodes persulcatus, whereas the western subtype is transmitted by Ixodes ricinus and causes Central European encephalitis (CEE). CEE can occur throughout much of Europe. Of the two subtypes, RSSE is the more severe infection, having a mortality of up to 25% in some outbreaks, whereas mortality in CEE seldom exceeds 5%. The incubation period is 7 to 14 days. Infection usually presents as a mild, influenza-type illness or as benign, aseptic meningitis, but may result in fatal meningoencephalitis. Fever is often biphasic, and there may be severe headache and neck rigidity, with transient paralysis of the limbs, shoulders or less commonly the respiratory musculature. A few patients are left with residual paralysis. Although the great majority of TBE infections follow exposure to ticks, infection has occurred through the ingestion of infected cows' or goats' milk. An inactivated TBE vaccine is currently available in Europe and Russia.

Venezuelan equine encephalitis (VEE) is an alphavirus and causes encephalitis in horses and humans and is an important veterinary and public health problem in Central and South America. Occasionally, large regional epizootics and epidemics can occur resulting in thousands of equine and human infections. Epizootic strains of VEE virus can infect and be transmitted by a large number of mosquito species. The natural reservoir host for the epizootic strains is not known. A large epizootic that began in South America in 1969 reached Texas in 1971. It was estimated that over 200,000 horses died in that outbreak, which was controlled by a massive equine vaccination program using an experimental live attenuated VEE vaccine. There were several thousand human infections. A VEE epidemic occurred in the fall of 1995 in Venezuela and Colombia with an estimated 90,000 human infections. Infection of man with VEE virus is less severe than with EEE and WEE viruses, and fatalities are rare. Adults usually develop only an influenza-like illness, and overt encephalitis is usually confined to children. Effective VEE virus vaccines are available for equines.

Enzootic strains of VEE virus have a wide geographic distribution in the Americas. These viruses are maintained in cycles involving forest dwelling rodents and mosquito vectors, mainly Culex (Melanoconion) species. Occasional cases or small outbreaks of human disease are associated with these viruses, the most recent outbreaks were in Venezuela in 1992, Peru in 1994 and Mexico in 1995-96.

In various embodiments, the method of the present invention includes administering an effective amount of one or more compositions including ribavirin. For purposes of the present invention, ribavirin is a nucleoside analog with antiviral activity. The chemical name of ribavirin is 1-β-D-ribofuranosyl-1H-1,2,4-triazole-3-carboxamide. Ribavirin has the empirical formula C₈H₁₂N₄O₅ and the molecular weight is 244.21.

In various embodiments, ribavirin exhibits the following structure:

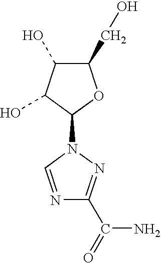

Ribavirin is available from various pharmaceutical companies. For example, a ribavirin composition is available as REBETOL® from Schering Plough Corporation, New Jersey.

In various embodiments, the present invention also includes pharmaceutically acceptable salts of ribavirin. Some examples of pharmaceutically acceptable salts include those salt-forming acids and bases that do not substantially increase the toxicity of the compound. Some examples of suitable salts include salts of alkali metals such as magnesium, potassium and ammonium. Salts of mineral acids such as hydrochloric, hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g. p-toluenesulfonic acids, and the like.

An effective amount of ribavirin as used herein is that amount effective to achieve the relief or palliation of symptoms, condition and/or diseases associated with arbovirus (e.g., WNV, SLE, Japanese encephalitis virus, etc.). Preferably, ribavirin is administered in an amount that inhibits the growth or replication of the arbovirus.

The minimal dose of ribavirin for a human is the lowest dose that achieves the desired result. For example, ribavirin is administered at a dose of from about 300 mg to about 3600 mg/day. Preferably, ribavirin is administered in an amount of 1200 mg as an initial dose, then 600 mg every 6 hours for 10 days.

Maximal dose for a human is the highest dosage that does not cause undesirable or intolerable side effects. For example, a maximal dose is a dose lower than 20-200 mg/kg (estimated human equivalent of 1.67-16.7 mg/kg), which is known to be mutagenic in mice. In determining the maximal and minimal dose, the practitioner is guided by skill and knowledge in the field, and the present invention includes without limitation dosages that are effective to achieve the described antiviral effect.

Administering ribavirin can be accomplished in a variety of ways. In cultured cellular or tissue systems, ribavirin can be administered by contacting the cells or tissue directly with an effective amount of ribavirin. In humans, ribavirin can be administered orally or enterally which is the preferred route of delivery. Formulations such as tablets, capsules, pills, troches, elixirs, suspensions, syrups, wafers, chewing gum and the like can be employed to provide ribavirin.

Ribavirin can also be administered by the parenteral route. For example, ribavirin can be administered intravenously (e.g., intravenous injection), intramuscularly, and/or subcutaneously. Intravenous administration can be accomplished by mixing ribavirin in a suitable pharmaceutical carrier (vehicle) or excipient as understood by practitioners in the art.

The present invention includes methods of treating arbovirus (e.g., WNV SLE, Japanese encephalitis virus, etc.) in an animal suffering therefrom comprising administering to the animal an effective amount of one or more compositions comprising interferon.

As used in this application, the term “interferon” includes recombinantly engineered and/or purified interferon α, β, or γ or combinations thereof. The term “interferon-alfa” as used herein means the family of highly homologous species-specific proteins that inhibit viral replication and cellular proliferation and modulate immune response. Typical suitable interferon-alfas include, but are not limited to, recombinant interferon alfa-2b, such as Intron-A® interferon available from Schering Corporation, Kenilworth, N.J., recombinant interferon alfa-2a, such as Roferon® interferon available from Hoffmann-La Roche, Nutley, N.J., recombinant interferon alpha-2c, such as Berofor® alpha 2 interferon available from Boehringer Ingelheim Pharmaceutical, Inc., Ridgefield, Conn., interferon alpha-n1, a purified blend of natural alfa interferons, such as Sumiferon® available from Sumitomo, Japan or as Wellferon® interferon alpha-n1 (INS) available from the Glaxo-Wellcome Ltd., London, Great Britain, or a consensus alpha interferon, such as those described in U.S. Pat. Nos. 4,897,471 and 4,695,623 (especially Examples 7, 8 or 9 thereof, herein incorporated by reference) and the specific product available from Amgen, Inc., Newbury Park, Calif., or interferon alfa-n3 which is a mixture of natural alfa interferon species derived from human leukocytes under the name ALFERON N Injection®, available from Hemispherx Biopharma, based in Philadelphia.

In various embodiments, the use of interferon alfa-2a or alpha 2b is preferred. The manufacture of interferon alpha 2b is described in U.S. Pat. No. 4,530,901 (herein incorporated by reference).

In various embodiments, peglylated interferon alfa can also be used. The term “pegylated interferon alfa” as used herein means polyethylene glycol modified conjugates of interferon alfa, preferably interferon alfa-2a and -2b. The preferred polyethylene-glycol-interferon alfa-2b conjugate is PEG₁₂₀₀₀-interferon alfa 2b. The phrases “12,000 molecular weight polyethylene glycol conjugated interferon alpha” and “PEG₁₂₀₀₀-IFN alfa” as used herein mean conjugates such as are prepared according to the methods of International Application No. WO 95/13090 and containing urethane linkages between the interferon alfa-2a or -2b amino groups and polyethylene glycol having an average molecular weight of 12000. The preferred PEG₁₂₀₀₀-interferon alfa-2b is prepared by attaching a PEG polymer to the epsilon amino group of a lysine residue in the IFN alfa-2b molecule. A single PEG]₂₀₀₀ molecule is conjugated to free amino groups on an IFN alfa-2b molecule via a urethane linkage. This conjugate is characterized by the molecular weight of PEG₁₂₀₀₀ attached. The PEG 12000-IFN alfa-2b conjugate is formulated as a lyophilized powder for injection. The objective of conjugation of IFN alfa with PEG is to improve the delivery of the protein by significantly prolonging its plasma half-life, and thereby provide protracted activity of IFN alfa.

Other pegylated interferon alfa conjugates can be prepared by coupling an interferon alfa to a water-soluble polymer. A non-limiting list of such polymers include other polyalkylene oxide homopolymers such as polypropylene glycols, polyoxyethylenated polyols, copolymers thereof and block copolymers thereof. As an alternative to polyalkylene oxide-based polymers, effectively non-antigenic materials such as dextran, polyvinylpyrrolidones, polyacrylamides, polyvinyl alcohols, carbohydrate-based polymers and the like can be used. Such interferon alfa-polymer conjugates are described in U.S. Pat. Nos. 4,766,106, 4,917,888 (herein incorporated by reference), European Patent Application No. 0 236 987, European Patent Application Nos. 0510 356, 0 593 868 and 0 809 996 (pegylated interferon alfa-2a) and International Publication No. WO 95/13090.

Pharmaceutical composition of pegylated interferon alfa suitable for parenteral administration may be formulated with a suitable buffer, e.g., Tris-HCl, acetate or phosphate such as dibasic sodium phosphate/monobasic sodium phosphate buffer, and pharmaceutically acceptable excipients, e.g., sucrose, carriers, e.g., humanor recombinant plasma albumin, tonicity agents, e.g. NaCl, preservatives, e.g., thimerosol, cresol or benyl alcohol, and surfactants, e.g., tweens or polysorabates in sterile water for injection. The pegylated interferon alfa-may be stored as lyophilized powders under a refrigeration at 2°-8° C. The reconstituted aqueous solutions are stable when stored between 2° and 8° C. and used within 24 hours of reconstitution. See for example U.S. Pat. Nos., 4,492,537; 5,762,923 and 5,766,582 (herein incorporated by reference). The reconstituted aqueous solutions may also be stored in prefilled, multi-dose syringes such as those useful for delivery of drugs such as insulin. Typical suitable syringes include systems comprising a prefilled vial attached to a pen-type syringe such as the NOVOLET® Novo Pen available from Novo Nordisk, as well as prefilled, pen-type syringes which allow easy self-injection by the user. Other syringe systems include a pen-type syringe comprising a glass cartridge containing a diluent and lyophilized pegylated interferon alfa powder in a separate compartment.

When the pegylated interferon-alfa administered is a pegylated interferon alfa-2b, the therapeutically effective amount of pegylated interferon alfa-2b administered during the treatment in accordance with the present invention is in the range of about 0.1 to 9.0 micrograms per kilogram of pegylated interferon alfa-2b administered per week, in single or divided doses, preferably once a week (QW) or twice a week (BIW), preferably in the range of about 0.1 to about 9.0 micrograms per kilogram of pegylated interferon alfa-2b administered once a week (QW) or in the range of about 0.05 to about 4.5 micrograms per kilogram of pegylated interferon alfa-2b administered twice a week (BIW), or is in the range of about 0.5 to about 3.0 micrograms per kilogram of pegylated interferon alfa-2b administered per week, preferably in the range of about 0.5 to about 3.0 micrograms per kilogram of pegylated interferon alfa-2b administered once a week (QW) or in the range of about 0.25 to about 1.5 micrograms per kilogram of pegylated interferon alfa-2b administered twice a week, or is in the range of about 0.75 to about 1.5 micrograms per kilogram of pegylated interferon alfa-2b administered per week, most preferably is in the range of about 0.75 to about 1.5 micrograms per kilogram of pegylated interferon alfa-2b administered once a week or about 0.375 to about 0.75 micrograms per kilogram of pegylated interferon alfa-2b administered twice a week.

When the pegylated interferon-alfa administered to pediatric patients is a pegylated interferon alfa-2b, the therapeutically effective amount of pegylated interferon alfa-2b administered during the treatment in accordance with the present invention is in the range of about 0.1 to 9.0 micrograms per kilogram of pegylated interferon alfa-2b administered per week, in single or divided doses, preferably once a week (QW) or twice a week (BIW), more preferably about 0.1 to about 9.0 micrograms per kilogram of pegylated interferon alfa-2b administered once a week (QW), or about 0.05 to about 4.5 micrograms per kilogram of pegylated interferon alfa-2b administered per week, in single or divided doses, preferably once a week (QW) or twice a week (BIW), more preferably about 0.05 to about 4.5 micrograms per kilogram of pegylated interferon alfa-2b administered once a week, or preferably about 0.75 to about 3.0 micrograms per kilogram of pegylated interferon alfa-2b administered in single or divided doses, preferably once a week (QW) or twice a week (BIW), more preferably about 0.75 to about 3.0 micrograms per kilogram of pegylated interferon alfa-2b administered once a week or about 0.375 to about 1.5 micrograms per kilogram of pegylated interferon alfa-2b administered twice a week, and most preferably about 2.25 to about 2.6 micrograms per kilogram of pegylated interferon alfa-2b administered once a week or about 1.1 to about 1.3 micrograms per kilogram of pegylated interferon alfa-2b administered twice a week (BIW).

When the pegylated interferon-alfa administered is a pegylated interferon alfa-2a, the therapeutically effective amount of pegylated interferon alfa-2a administered in accordance with the present invention, is in the range of about 50 micrograms to about 500 micrograms QW, preferably about 150 micrograms to about 250 micrograms QW or the effective amount is in the range of about 50 micrograms to about 250 micrograms twice a week, preferably about 100 micrograms to about 125 micrograms twice a week.

When the pegylated interferon-alfa administered to a pediatric patient is a pegylated interferon alfa-2a, the therapeutically effective amount of pegylated interferon alfa-2a administered in accordance with the present invention, is in the range of about 50 micrograms to about 500 micrograms QW, preferably about 300 micrograms to about 375 micrograms QW or the therapeutically effective amount of pegylated interferon alfa-2a administered to a pediatric patient is in the range of about 50 micrograms to about 250 micrograms twice a week, preferably about 150 micrograms to about 190 micrograms once a week.

While the mechanism of antiviral activity of interferon is still unknown, it is believed that the anti-viral activity of interferon is not caused directly by interferon, rather it is caused by proteins that are induced or signaled by interferon. This signaling pathway activates the immune response of the cell.

For example, interferon alpha-2b inhibited viral replication at a relatively low concentration (5.9 units/mL) when applied after infection of green monkey kidney cells with WNV. Ribavirin suppressed viral replication at a concentration of 500 μM when applied after infection. A cytotoxic effect of ribavirin occurred at a concentration of 600-1000 μM. Thus, interferon alpha-2b possesses greater activity in-vitro than ribavirin, with a potentially greater therapeutic ratio in humans. A dose of 3 million units of interferon alpha-2b in humans provides serum levels of 10 units/mL after 8 hours, and daily doses of 3 million units yield serum levels of 20-30 units/mL, which are above the concentration required for in-vitro efficacy against arborvirus (e.g., WNV). Although systemic administration of interferon alpha-2b produces low levels in cerebrospinal fluid and brain, beneficial effects against encephalitis may occur through suppression of viremia and/or enhancement of cell mediated immunity systemically and in the central nervous system.

An effective amount of interferon as used herein is that amount effective to achieve the relief or palliation of symptoms, condition and/or diseases associated with arborvirus. Preferably, interferon is administered in an amount that indirectly inhibits the growth or replication of the arborvirus by mediating the immune response.

The minimal dosage of interferon for a human is the lowest dose which achieves the desired result. For example, interferon alpha 2a or alpha-2b is administered at a dose of from about 1.5 million units to about 10 million units/day. Preferably, interferon alpha 2a or alpha-2b is administered in an amount of 3 million units as an initial dose, then 3 million units every 12-24 hours, typically for 7-14 days.

In various embodiments, the pegylated interferon-alfa 2b can be administered to adults at a dosage of about 6-10 million subcutaneously QW for treatment of arbovirus infections.

In various embodiments, interferon alfa-n3 is administered in an amount of 3 million units as an initial dose, then 3 million units every 12-24 hours, typically for 7-14 days.

Maximal dosage for a human is the highest dosage that does not cause undesirable or intolerable side effects. For example, a maximal dose is a dose lower than 15 and 30 million units/kg (estimated human equivalent of 5 and 10 million nits/kg) which was shown to have abortifacient effects in pregnant rhesus monkeys. However, in determining the maximal and minimal dose, the practitioner is guided by skill and knowledge in the field, and the present invention includes without limitation dosages that are effective to achieve the described antiviral effect.

The interferon compounds, in various embodiments of the present invention, may be formulated for parenteral administration (e.g. by injection, for example bolus injection or continuous infusion), subcutaneous injection, or intramuscular injection and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing an/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

In various embodiments, an arborvirus dosing kit is provided, which comprises an initial bolus unit dose of interferon by injection at a dose of 3 million units in a suitable volume of diluent (e.g. D5W, NS, etc.). The kit further comprises additional doses of interferon at 3 million units in unit dose form (e.g., ampoules, pre-filled syringes) for subcutaneous injections.

The interferon can be administered in a variety of ways. In cultured cellular or tissue systems, interferon can be administered by contacting the cells or tissue directly with an effective amount of interferon. In humans, interferon can be administered by the parenteral route. For example, interferon can be administered intravenously (e.g., intravenous injection), subcutaneously, intradermally, or by intramuscular injection. Intravenous administration can be accomplished by mixing interferon in a suitable pharmaceutical carrier (vehicle) or excipient as understood by practitioners in the art.

The present invention includes composition and methods of treating a human suffering from a meningitis, encephalitis, meningo-encephalitis or encephalomyelitis caused by an arbovirus, comprising parenterally administering to the human an effective amount of interferon alone or in combination with ribavirin. For example, combined or coordinated systemic administration of interferon and ribavirin is also contemplated in various embodiments of the invention. Preferred combined systemic administration includes oral administration of ribavirin to humans in amounts from about 300 mg to about 3600 mg/day and parenteral administration of interferon in an amount from about 1.5 million units to about 10 million units/day or pegylated interferon-alfa 6-10 million subcutaneously QW.

It is believed that combined administration of ribavirin and interferon alpha is additive or synergistic. Further, it appears that combined treatment increases therapeutic efficacy in tissue culture.

The methods of the present invention can be used in vivo, in vitro, and ex vivo, for example, in living animals as well as in cultured tissue, organ or cellular systems. Animals include mammals, for example, humans, as well as pet animals such as dogs and cats, laboratory mammals, such as rats and mice, and farm animals, such as pigs and horses and cows. Animals include poultry such as chickens and turkeys, and other birds such as pigeons and crows.

Tissues, as used herein, are an aggregation of similarly specialized cells that together perform certain special functions. Cultured cellular systems include any cells that can be infected with arborvirus (e.g., WNV), such as for example, blood cells, brain or kidney cells.

In vivo practice of the invention permits application in the prevention, relief or palliation of medical and veterinary, syndromes, symptoms, conditions or diseases associated with arborvirus (e.g., WNV). In particular, the method provides a means for protecting animals suffering from diseases or other conditions associated with or mediated by arborvirus (e.g., WNV). Such conditions or diseases include but are not limited to: infection, viremia, stiff neck headache, fever, myalgia, change of mental status, peripheral neurologic abnormalities, muscle weakness, rash, meningitis, encephalitis, encephalomyelitis, and/or meningoencephalitis.

The methods of the present invention are also applicable for prophylaxis or prevention of disease. Thus, an animal can be given dose(s) of ribavirin and/or interferon on a weekly or daily basis to prevent arborvirus (e.g., WNV) infection. Such dose(s), preferably, is the same or less than that used for treatment. Preferably, the animal is given preventative doses of ribavirin and/or interferon before exposure to arborvirus (e.g., WNV). In any event, in determining the preventative dose, the practitioner is guided by skill and knowledge in the field, and, in various embodiments, the present invention includes without limitation dosages that are effective to prevent arborvirus (e.g., WNV) infection.

Having now generally described the invention, the same may be more readily understood through the following reference to the following examples, which are provided by way of illustration and are not intended to limit the present invention unless specified.

### EXAMPLES

The examples below describe methods for preventing and treating WNV in vitro and in vivo.

### Example 1

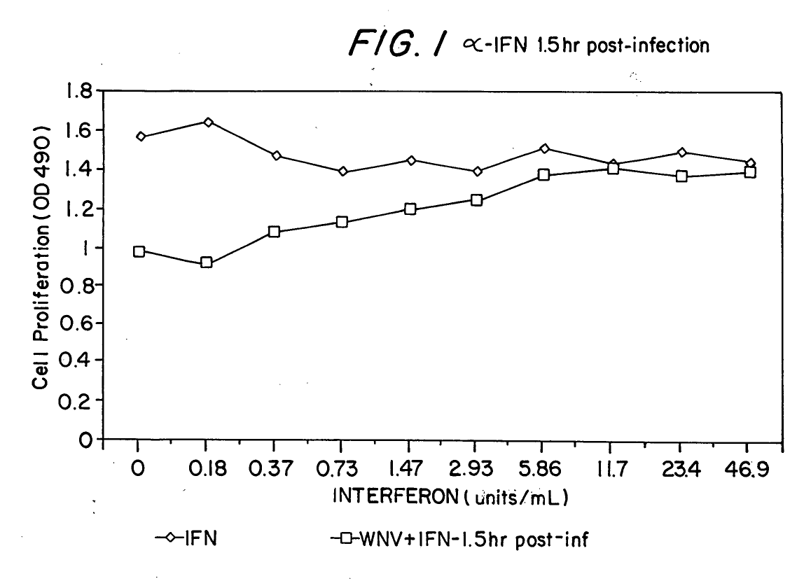

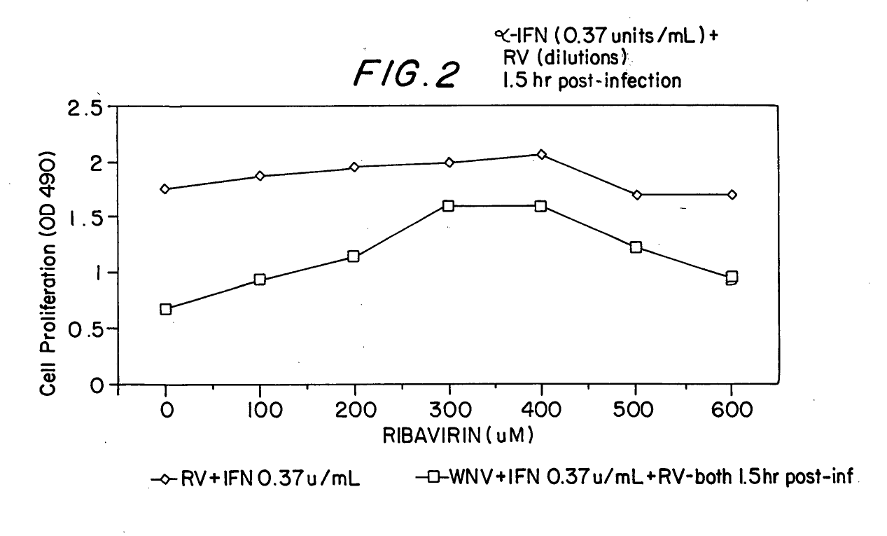

In vitro: Studies have been conducted utilized a bovine kidney cell monolayer infected with a strain of WNV isolated from mosquitoes and birds in Connecticut. Cytotoxicity was assayed by measuring the decrease in release of lactate dehydrogenase from infected cells, as compared with uninfected controls. Experiments were conducted with the addition of serial dilutions of ribavirin, interferon alpha-2b, or both, prior to, or after, infection of cells by WNV. The results are shown in Table 1 and FIGS. 1 and 2.

TABLE 1
Suppression of West Nile virus (WNV) in Vero cell
culture by ribavirin and interferon alpha-2b.

| | Mean* | | |
|---|---|---|---|
| | | O.D. 490 | |
| Treatment | Dose | value | +SD |
| Interferon (units/ml) | 6000 | 0.94b | 0.083 |
| applied 2 hours prior to | 3000 | 1.02c | 0.102 |
| infection of cells with WNV | 1500 | 1.09c | 0.050 |
| | 750 | 1.05c | 0.118 |
| | 375 | 1.08c | 0.073 |
| | 188 | 1.07c | 0.070 |
| | 0 | 0.71a | 0.058 |
| Interferon (units/ml) | 375.0 | 1.51a | 0.156 |
| applied 2 hours after | 94.0 | 1.44a | 0.033 |
| infection of cells with WNV | 23.4 | 1.43a | 0.123 |
| | 5.9 | 1.37ab | 0.220 |
| | 1.5 | 1.21abc | 0.006 |
| | 0.4 | 1.09acb | 0.021 |
| | 0 | 0.94c | 0.155 |
| Ribavirin (uM) applied | 0.6 | 1.06a | 0.268 |
| 2 hours prior to | 0.5 | 1.24b | 0.430 |
| infection of cells with WNV | 0.4 | 1.24b | 0.344 |
| | 0.3 | 1.11a | 0.265 |
| | 0.2 | 0.72c | 0.063 |
| | 0 | 0.57d | 0.074 |
| Interferon (0.37-1.5 units/ml) | 0.50 | 1.25bc | 0.215 |
| plus doses of ribavirin (uM) | 0.40 | 1.56ba | 0.193 |
| applied 2 hours after | 0.30 | 1.69a | 0.354 |
| infection of cells with WNV | 0.20 | 1.44ab | 0.360 |
| | 0.10 | 1.11dc | 0.223 |
| | 0.05 | 0.87d | 0.220 |
| | 0 | 0.78d | 0.168 |

*Means with the same letter within each treatment were not significantly different at p < 0.05 using Tukey HSD multiple comparison test.

Both ribavirin and interferon alpha-2b are active in vitro against WNV infection of bovine kidney cells. A relatively high concentration of ribavirin (400 uM), is protective or prophylactic against infection. A 400 uM concentration is equivalent to approximately 100 ug/ml: The concentration of ribavirin in human serum after 600 mg given every 6 hours is 1 ug/ml, 100-fold less than the in vitro inhibitory concentration. However, ribavirin is broadly concentrated intracellularly in vivo with subsequent phosphorylation and dephosphorylation. Thus, the relationship between in vitro inhibitory concentration, serum concentration, in vivo intracellular concentration, and ultimate in vivo antiviral effect can be predicted.

Interferon alpha-2b is both protective and therapeutic for WNV infected bovine kidney cells in vitro at relatively low concentrations, approximately 1-10 units/ml. A dose of 3 million units of interferon alpha-2b in humans provides serum levels of 10 units/ml after 8 hours, and daily doses of 3 million units yield serum levels of 20-30 units/ml, which are above the concentration required for in vitro efficacy against WNV. Although systemic administration of interferon alpha-2b produces low levels in cerebrospinal fluid and brain, beneficial effects on West Nile encephalitis may occur through suppression of viremia and/or lymphocyte activation, systemically and in the central nervous system.

Full protection of cell viability occurred when a combination of interferon alpha-2b, 1.47 units/ml, and ribavirin, 200-300 uM, was added 1.5 hours after infection of cells with WNV. Control cells infected with WNV, without addition of drug, showed loss of viability.

Ribavirin toxicities: The major toxicity of ribavirin is hemolysis due to accumulation of ribavirin triphosphate within erythrocytes leading to a decreased life span. Such accumulation occurs due to inability of erythrocytes to dephosphorylate the triphosphate. Inhibition of erythrocyte release from bone marrow occurs at high doses (30 mg/kg). Hemolysis is related to the dose and duration of therapy, and is reversible after discontinuation. A maximum of 20% reduction from baseline hematocrit occurred during effective therapy of Lassa fever with 4 gm/d for 4 days, followed by 1.5 gm/d for 6 days. Prophylactic use of ribavirin, 600 mg 4 times daily for Lassa fever contact caused a 10-12% reduction in baseline hemoglobin after 10 days. Ribavirin is teratogenic and should not be given during, or within 6 months of pregnancy. Bioavailability is increased in patients with renal dysfunction.

Interferon alpha-2b toxicities: A flu-like syndrome with fever, malaise and headache occur shortly after interferon alpha-2b administration. Longer term effects include depression and 35-40% reductions in peripheral neutrophile and platelet counts. Abnormal liver function is a relatively uncommon toxicity.

### Example 2

In vivo studies are contemplated. Adult patients, with WNV encephalitis documented by positive serum or cerebrospinal fluid WNV IgM antibody, and/or positive serum WNV neutralizing antibody, will be eligible for treatment if serum creatinine clearance is above 50 ml/min.

Therapy: Patients will be treated with oral ribavirin, 1200 mg, as an initial dose, followed by 600 mg every 6 hours. Treatment with interferon alpha-2b will be given initially as an intravenous dose of 3 million units, followed by a subcutaneous injection of 3 million units after 12 hours, and then every 24 hours. Therapy with both drugs will be initiated simultaneously and continued, if tolerated, for 10 days. If patients are unable to take oral medication, the contents of ribavirin capsules, 600 mg, will be dissolved in 30 ml of sterile water and given by naso-duodenal intubation. Care will be taken to ensure that the feeding tube passes beyond the stomach to allow full absorption of ribavirin, avoiding gastric hydrolysis. Doses of ribavirin will be given 30 minutes after enteric feeding, or after an oral meal to increase absorption.

Acetaminophen, 650 mg, will be given 30 minutes prior to each dose of interferon alpha-2b. The dose of 3 million units will be reconstituted from interferon alpha-2-b (Intron A) powder for injection, and will be given intravenously (first dose) and by subcutaneous injection (subsequent doses).

Patient Examination: Patients will be examined daily throughout therapy with recording of maximum temperature, level of consciousness, orientation, mental acuity, motor and sensory function, rash, organomegaly or other abnormal findings.

Laboratory and Radiologic Studies: Prior to initiation of therapy, cerebral computerized tomography or magnetic resonance imaging, electroencephalography, lumbar puncture, complete blood count, and hepatic/renal function tests will be done. Complete blood count and hepatic/renal function tests will be repeated daily during therapy. Radiologic studies and electroencephalography will be repeated as indicated. Lumbar puncture will be repeated at the end of therapy, and at other times as indicated clinically. Therapy will be discontinued if the peripheral absolute neutrophile count falls below 1000 per mm³; the peripheral platelet count falls below 50,000 per mm³; hepatic enzymes increase by 3-fold; or serum creatinine increases by 2-fold and is greater than 2.0 mg/dl.

Evaluation of Treatment Outcome: The most important evaluation of treatment is patient survival, because the mortality of prior central nervous system WNV infection in New York was 11%, and higher among patients with encephalitis. This will be supplemented by an evaluation of neurologic deficits at monthly intervals for 6 months and at one year.

### Example 3

In vivo studies with interferon alpha-2b: Interferon alpha-2b, as previously mentioned possesses greater activity in-vitro than ribavarin, with a potentially greater therapeutic ratio in humans. This is a pilot study of at least 10 patients designed to test tolerance of interferon alpha-2b in the treatment of central nervous system infection and its potential therapeutic effect prior to a subsequent controlled study. To initiate therapy at the earliest possible stage and before brain damage occurs, patients in this age group with any sign of central nervous system infection by WNV will be eligible.

Therapeutic Plan: Death due to WNV infection is universally due to meningitis which progress to encephalitis. Thus, therapeutic intervention should be limited to this population at risk. To initiate therapy at the earliest possible stage and before brain damage occurs, patients in this age group with any sign of central nervous system infection by WNV will be eligible. This is a pilot study of at least 10 patients designed to test tolerance of interferon alpha-2b in the treatment of central nervous system infection and its potential therapeutic effect prior to a subsequent controlled study.

Patient selection: Adult patients, with WNV meningitis or encephalitis will be treated in an open study after informed consent is obtained from the patient or appropriate surrogate. WNV infection must be documented by positive serum or cerebrospinal fluid WNV IgM antibody, and/or positive serum WNV neutralizing antibody. Because subcutaneous and intravenous interferon alpha-2b is FDA approved for treatment of hepatitis C infection, off-label use for WNV infection is allowed without further FDA or IRB approval. However, it is advisable that IRB approval be obtained for this protocol.

Therapy: Patients will be treated with an initial intravenous dose of 3 milllion units of interferon alpha-2b followed by a subcutaneous injection of 3 million units after 12 hours, and then every 24 hours. Therapy will be continued, if tolerated, for 14 days. Acetaminophen, 650 mg, will be given 30 minutes prior to each dose of interferon alpha-2b. The dose of 3 million units will be reconstituted from interferon alpha-2b (Intron A) powder for injection, provided by Schering-Plough Corporation.

Patient Examination: Patients will be examined daily throughout therapy with recording of maximum temperature, level of consciousness, orientation, mental acuity, motor and sensory function, rash, organomegaly or other abnormal findings.

Laboratory and Radiolopic Studies: Prior to initiation of therapy, cerebral computerized tomography or magnetic resonance imaging, electroencephalography, lumbar puncture, complete blood count, and hepatic/renal function tests will be done. Complete blood count and hepatic/renal function tests will be repeated daily during therapy. Radiologic studies and electroencephalography will be repeated as indicated. Lumbar puncture will be repeated at the end of therapy, and at other times as indicated clinically. Therapy will be discontinued if the peripheral absolute neutrophil count falls below 1000 cells per mm3; the peripheral platelet count falls below 50,000 per mm3; hepatic enzymes increase by 3-fold; or serum creatinine increases by 2-fold or to greater than 2.5 mg/dL. At onset of therapy, and after 7 and 14 days, 10 mL of blood will be obtained, and serum frozen at -20° C for subsequent study of cell mediated immunity against WNV. Two mL of CSF from all lumbar punctures will be frozen for the same purpose.

Evaluation of Outcome: Unless WNV encephalitis occurs with sufficient frequency to allow a controlled, blinded study of therapy with interferon alpha-2b, only sporadic patients will be treated. Because the mortality of WNV encephalitis in North America has been approximately 20%, the most important evaluation of sporadic therapy is survival. This will be supplemented by an evaluation of neurologic deficits at monthly intervals for 6 months and at one year.

### Example 4

In order to assess whether interferon alpha-2b was effective for the treatment of WNV meningo-encephalitis, a randomized, unblinded, multi-institutional clinical study was conducted using the protocol developed in Example 3. The dose and route of administration of interferon alpha-2b was 3 million units intravenously (IV) initially; then 3 million units subcutaneously 12 hours later, then 3 million units subcutaneously every 24 hours for up to 14 days of therapy.

Patient Enrollment:

During the summers of 2002-2003, patients with clinical and epidemiologic evidence of WNV meningo-encephalitis were enrolled. Patients were randomly assigned into two groups: group 1 received interferon alpha-2b for two weeks (6 million units initially-3 million units intravenously (IV) initially; then 3 million units subcutaneously 12 hours later) followed by 3 million units daily plus standard supportive therapy; group 2 did not receive interferon, but received standard supportive therapy alone. Treatment was initiated prior to the results of WNV serologic studies. Patients with serologically-proven WNV infection and follow-up examination after 3 weeks were included in the outcome analysis.

Interferon Dose Regimen

IV interferon alpha-2b was given at a dose of 3 million units initially; then 3 million units by subcutaneous injection 12 hours later, followed by 3 million units subcutaneously every 24 hours to complete up to 14 days of therapy if tolerated. The interferon alpha-2b in the clinical study was given by the intravenous and subcutaneous route and not by intracranial administration.

Clinical Evaluation of Outcome:

19 patients were randomized to each group. Among the interferon treated patients, 2 were seronegative, 1 was lost to follow up, and 1 died within the first 36 hours. Among the patients that did not receive interferon, 4 patients were seronegative, 4 were lost to follow up, and three withdrew. Thus, 15 interferon treated and 8 patients that did not receive interferon were eligible for analysis. The evaluation of outcome was determined by assessment of neurologic status at enrollment, using the National Institute of Health Stroke Scale (N.I.H.S.S.), which provides an initial functional score of 0 or negative number (lowest function) to 42 (highest function). The neurologic examination is scored with the N.I.H.S.S. to quantify the degree of neurologic dysfunction from the WNV infection. Neurologic dysfunction is the result of active viral invasion of the CNS causing inflammation and ultimate cell destruction. Resolution of neurologic dysfunction is a sign of resolving infection. A high N.I.H.S.S. score correlates with a large neurologic dysfunction. In the clinical study, this scale was used to assess whether CNS infection, such as a meningitis or encephalitis, is resolving. Subsequent examinations help quantify the progress of the patient. Typically, the patient is assessed and scored as follows:

Level of Consciousness

0=Alert, keenly responsive

1=Drowsy, arousable by minor stimulation to obey, answer, or respond

2=Responds only with reflex motor or autonomic effects, or totally unresponsive

Level of Consciousness Question: Patients are Asked the Month and Their Age

0=Both correct or language barrier

1=One correct

2=Both incorrect, or unable to respond

Level of Consciousness Command: The Patient is Asked to Close the Eve and the Hand

0=Both correct or language barrier

1=One correct

2=Both incorrect, or unable to respond

Best Visual: Test Vision in Each Field to Finger Movement Simultaneously

0=Normal or old blindness

1=Asymmetry or partial hemianopia

2=Complete hemianopia

3=Bilateral hemianopia or coma

Best Gaze

0=Full range of eye movements

1=Partial gaze palsy or isolated nerve palsy

2=Forced deviation or total gaze paresis not overcome by Doll's eye maneuver

Facial Weakness

0=None or sedated

1=minor Oust loss of naso-labial fold)

2=Partial (lower half of the face)

3=Complete (all half involved) or coma

Best Motor Left Arm: The Patient Holds the Arm Outstretched at 90 Degrees.

0=Limb holds 90 degrees for full 10 seconds, effusion or amputation

1=Limb holds 90-degree position, but drifts before full 10 seconds

2=Limb cannot hold 90-degree position for full 10 seconds, some effort against gravity

3=Limb falls, no effort against gravity

4=No movement

Best Motor Right Arm: The Patient Holds the Arm Outstretched at 90 Degrees

0=Limb holds 90 degrees for full 10 seconds, effusion or amputation

1=Limb holds 90-degree position, but drifts before full 10 seconds

2=Limb cannot hold 90-degree position for full 10 seconds, some effort against gravity

3=Limb falls, no effort against gravity

4=No movement

Best Motor Left Leg: The Patient Elevates the Leg at 30 Degrees for 5 Seconds.

0=Leg holds 30-degree position for 5 seconds, effusion or amputation

1=Leg falls to intermediate position by end of 5 seconds

2=Leg falls to bed by 5 seconds, some effort against gravity

3=Leg falls to bed immediately, no resistance against gravity

4=No movement

Best Motor Right Leg: The Patient Elevates the Leg at 30 Degrees for 5 seconds.

0=Leg holds 30-degree position for 5 seconds, effusion or amputation

1=Leg falls to intermediate position by end of 5 seconds

2=Leg falls to bed by 5 seconds, some effort against gravity

3=Leg falls to bed immediately, no resistance against gravity

4=No movement

Limb Ataxia: Finger-To-Nose and Heel-To-Shin Test

0=Absent (no movement of limb), can not be examined

1=Ataxia present in one limb

2=Ataxia present in two limbs

Sensory: Pin Prick. If Level of Consciousness is Impaired, Score Only if a Grimace or Asymmetric Withdrawal is Present.

0=Normal, sedated or amputation

1=Mild to moderate. Patient feels pin prick less sharp, but is aware of being touched

2=Severe to total sensation loss, not aware of being touched Neglect

0=No neglect or sedated

1=Visual, tactile, or auditory hemi-inattention

2=Profound hemi-inattention to more than one modality

Dysarthria

0=Normal

1=Mild to moderate slurring of words, can be understood

2=Speech slurred, unintelligible

Best Language: Standard Pictures are Named

0=Normal

1=Mild to moderate naming errors, word-finding errors, or paraphasias. Impairment of communication by either comprehension or expression.

2=Severe: fully developed Broca's (expressive) or Wemicke's (receptive) aphasia 3=Mute or global aphasia or coma.

The N.I.H.S.S. in this trial was determined again at 1, 2, and 3 weeks after enrollment. The difference between the functional score at enrollment and after 3 weeks was determined for each group. The effectiveness of interferon alpha-2b was determined by a comparison of the mean change in N.I.H.S.S. scores among interferon treated patients and patients that did not receive interferon.

Results:

The change in N.I.H.S.S. for the interferon treated patients at 3 weeks after enrollment is shown in Table 2 and untreated patients is shown in Table 3.

TABLE 2
Neurological Score After 3 Weeks For Patients Given Interferon

| | Neurological | Interferon Dose | | |
|---|---|---|---|---|
| Patient | Score | And Length Of | Age | Serum |
| No. | (0 to +23) | Treatment | (M/F) | +/− |
| 1 | +5 | 14 | 74 M | + |
| 2 | 0 | 14 | 53 M | + |
| 3 | +6 | 10 | 81 F | + |
| 4 | +7 | 12 | 81 M | + |
| 5 | +5 | 14 | 40 M | + |
| 6 | +20 | 14 | 73 F | + |
| 7 | +20 | 14 | 75 M | + |
| 8 | +6 | 10 | 83 F | + |
| 9 | +2 | 14 | 45 M | + |
| 10 | +8 | 10 | 76 F | + |
| 11 | +12 | 14 | 47 M | + |
| 12 | +23 | 14 | 85 F | + |
| 13 | +7 | 14 | 66 M | + |
| 14 | +12 | 14 | 25 M | + |
| 15 | +11 | 14 | 73 M | + |
| MEAN | +9.6 | 13.1 | Age 65.1 | All |
| | (p = .008) | | | positive |

TABLE 3
Neurological Score After 3 Weeks
For Patients Not Given Interferon

| Patient | Score | Age | Serum |
|---|---|---|---|
| No. | (−1 to +8) | (M/F) | +/− |
| 1 | +6 | 74 M | + |
| 2 | +2 | 65 F | + |
| 3 | +3 | 68 M | + |
| 4 | +1 | 57 F | + |
| 5 | +1 | 71 M | + |
| 6 | +4 | 80 M | + |
| 7 | −1 | 62 M | + |
| 8 | +8 | 62 M | + |
| Mean | +3.0 | Age 67.4 | All positive |
| | (p = .008) | | |

Changes designated with a plus sign indicate the degree of improvement, and by a minus sign indicate the degree of deterioration, on a scale of 0-42. The mean improvement among 15 treated patients was +9.6 and among 8 untreated patients, +3.0. This difference is statistically significant (p=0.008 by 2-tailed Fisher's randomization test) with a confidence of greater than 95%. Alpha-2b interferon was given for an average of 13.1 days. The most common adverse events were elevation of serum transaminase and neutropenia. Grade 3 hepatotoxicity occurred in 37.5% of the patients, and grade 3 neutropenia in 31 % of the patients. These events resolved with drug cessation.

Conclusion:

Treatment of patients with West Nile viral meningitis or encephalitis with interferon alpha-2b by the IV route for up to 14 days results in a significantly improved neurologic functional status and survival after three weeks, as compared to patients that did not receive the interferon alpha-2b.

Thus, following my protocol described in Example 4, it is shown that interferon alpha-2b IV, initially, followed by subcutaneous injections for the successful treatment of WNV meningo-encephalitis. The interferon alpha-2b is not administered by the intracranial route, but by the IV route at a dose of 3 million units IV initially; then 3 million units subcutaneously 12 hours later, then followed by 3 million units subcutaneously every 24 hours for up to 14 days of therapy.

### Example 5

Effect of Interferon-2b Therapy on St. Louis Viral Meningoencephalitis: Clinical and Laboratory Results of a Pilot Study

Interferon (IFN)-α2b is active in vitro against at least 3 members of the Flaviviridae family: hepatitis C virus, Japanese encephalitis virus, and West Nile virus. All 3 viruses are structurally and genetically related to SLE virus.

An outbreak of St. Louis meningoencephalitis due to SLE virus occurred in northeastern Louisiana during the summer of 2001. Thirteen patients were admitted to the same hospital during the first 3 weeks of August. Three became quadriplegic and required ventilatory assistance during the first week of hospitalization. Because of the taxonomic, serologic, and clinical association between SLE viral disease and West Nile viral disease, as well as the known in vitro activity of IFN-α2b against West Nile virus, we sought to determine whether early initiation of IFN-α2b therapy for central nervous system (CNS) disease due to SLE virus would be safe and potentially beneficial, by conducting an open, uncontrolled interventional trial.

Patients, Materials, and Methods

Patients were included in the present study if they were admitted to the hospital with fever and/or severe headache and cerebrospinal fluid (CSF) pleocytosis. Inclusion in the study initially required documentation of the presence of IgM antibody against SLE virus in serum or CSF. With progression of the outbreak of St. Louis meningoencephalitis, patients with the aforementioned clinical and laboratory findings were included in the study pending the results of serologic testing. If such results were negative, patients were withdrawn from the study. Additional signs noted at, or after, admission to the hospital were nystagmus, diplopia, ophthalmoplegia, respiratory insufficiency, tremor, ataxia, peripheral weakness, confusion, or decreased level of consciousness. Thus, no distinction between meningitis and meningoencephalitis was attempted for the purpose of inclusion of patients in the trial.

Because advanced infection of the CNS with SLE virus produces neuronal degeneration, patients were excluded from the trial if they were seen with clinical, electroencephalographic, or computed tomographic evidence of severe brain damage . For all patients, diagnosis of infection with SLE virus was established by detection of IgM flavivirus antibody in serum or CSF, by use of an IgM indirect fluorescent antibody assay at the Louisiana State Department of Health Laboratories. Assays for the detection of antibody directed against California virus and avirus groups were performed as well. Selected patients were evaluated by the αvision of Vector-Borne Infectious Diseases, Centers for Disease Control and Prevention (CDC; Fort Collins, CO), by use of the IgM capture ELISA and the plaque reduction neutralization test.

Patients were treated with an initial intravenously administered dose of 3 million U of IFN-α2b, followed by subcutaneous injection of 3 million U of IFN-α2b 12 h later and then every 24 h for 14 days. The dose that was chosen was that which would result in ˜30 U of IFN-α2b/mL of blood, exceeding the lowest in vitro inhibitory concentration of IFN-α2b against SLE virus.

Therapy was temporarily withheld if the absolute neutrophil count decreased to <1000 neutrophils/mm3. IFN-α2b was provided, as Intron A, by Schering-Plough. Treatment of St. Louis meningoencephalitis with IFN-α2b was approved by the US Food and Drug Administration, under Investigational New Drug #9984.

Patients were examined at admission to the hospital and daily thereafter by one of the authors (L.L.T.). A neurologic function score was determined weekly. The neurologic capacities assessed and the possible number of points assigned to each neurologic capacity were as follows: asymptomatic status (1 or 0 points); walking, talking, thinking, and swallowing (2, 1, or 0 points per each capacity assessed); and unassisted respiration (1 or 0 points).

Asymptomatic patients received a neurologic function score of 10. Patients who had quadriplegia and required ventilatory assistance received a score of 0. These neurologic function scores and other pertinent clinical and laboratory data were recorded. The hospital and follow-up outpatient records of all patients were reviewed retrospectiyely in the study. The same information was obtained from the records of the first 13 untreated patients who were admitted to the hospital with SLE virus infection before the availability of IFN-α2b and from 4 patients who declined treatment. These 17 patients routinely had been evaluated by the same physician, by use of the aforementioned neurologic function score, which was recorded within notes on patient progress. Severe neurologic complications and their definitions were as follows: “quadriplegia” was defined as absence of muscle strength in all extremities, “quadriparesis” was defined as muscle strength of 1-3 (of a highest possible strength of 5) in all extremities, and “respiratory insufficiency” was defined as a requirement for ventilatory assistance.

SLE virus strain LA-01-5981 was cultured from Culex quinquefasciatus collected in Monroe, Louisiana, on 4 September 2001, and provided by H. M. Savage of the CDC. The in vitro system for the susceptibility assay was identical to that previously reported for West Nile virus, with the exception of substitution of baby hamster kidney cells for Vero cells to achieve adequate replication of the SLE virus.

Informed consent was obtained from each treated patient or his/her guardian. Human experimentation guidelines of the US Department of Health and Human Services were followed.

Results

Thirty-two patients were considered to have St. Louis viral meningoencephalitis. All had IgM flavivirus antibody detected by indirect fluorescent assay, and the results of all tests for California and virus group antibody were negative. Four patients studied by the CDC demonstrated cross-reactions between SLE viral antigens and West Nile viral antigens. However, plaque reduction neutralization assays yielded higher titers against SLE virus (range, 1:40-1:2560) than against West Nile virus (range, <1:10-1:40). The clinical symptoms and signs of St. Louis viral meningoencephalitis included fever (temperature, ≧38.3° C.) in 24 patients, headache in 25, gastrointestinal symptoms in 17, nuchal rigidity in 11, ataxia in 7, tremor in 7, myalgia in 6, diplopia in 2, and ophthalmoplegia in 1 patient.

All patients had CSF pleocytosis. The range of total white blood cell (WBC) counts in the CSF was 7-688 WBCs/mm3, the percentage of neutrophils noted was 1%-87%, and the percentage of lymphocytes noted was 9%-81%. The range of protein concentrations in the CSF was 31-149 mg/dL, and that of the glucose concentrations was 44-187 mg/dL.

Fifteen patients were treated with IFN-α2b for 2 weeks, starting on days 1-4 after admission to the hospital (mean time to initiation of treatment with IFN-α2b, 1.93 days). The full course of therapy, including treatment interruptions resulting from development of granulocytopenia or hepatitis, required 14-24 days (mean, 18.8 days). Of the untreated patients, 13 were admitted before the onset of the IFN-α2b protocol. Four other untreated patients chose not to be treated. One untreated patient experienced a relapse after discharge from the hospital, and was readmitted and treated.

Nine untreated patients were female, and 8 were male. Ten were white, 5 were black, and 2 were Latino. The mean age of the untreated patients was 49.5 years. Of the treated patients, 7 were female and 8 were male; 13 were white and 2 were black. The mean age of the treated patients was 44.5 years. Important underlying disease was rare in both groups. Hypertension, which was the most frequently occurring abnormality, was noted in 5 untreated patients and in 2 treated patients. Diabetes mellitus was present in 2 patients (1 treated patient and 1 untreated patient). Two untreated patients and 1 treated patient had atrial fibrillation. Migraine headache, hyperthyroidism, and cardiomyopathy were each noted once among 3 separate patients.

One patient who had coma and quadriplegia and who was ventilator dependent for 2 weeks was inadvertently enrolled in the trial after transfer to the study site. Data on the course of her disease were excluded from data analysis.

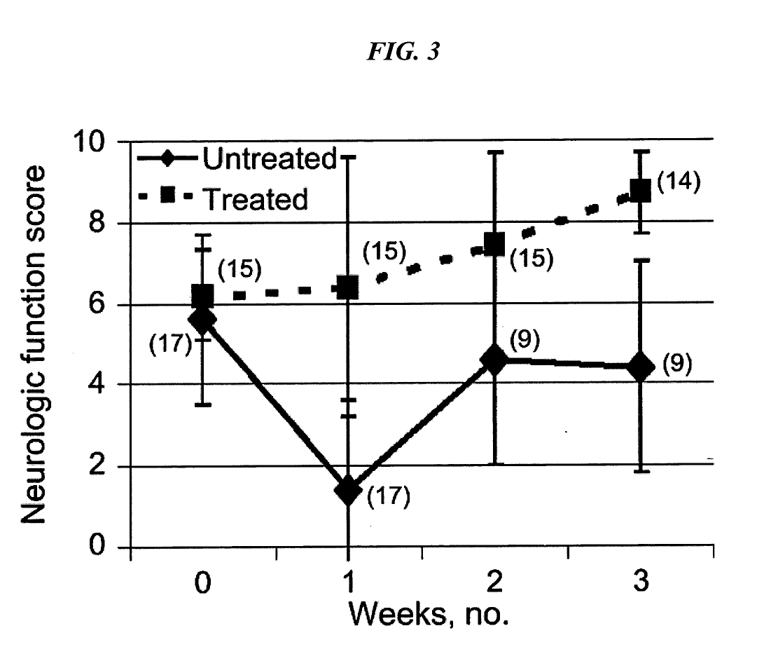

The mean neurologic function score of 15 untreated patients at admission to the hospital (i.e., at “week 0”) was 5.6 (SD, ±2.1), and that of the treated patients was 6.2 (SD, ±1.1), of a possible range of 0-10. The mean neurologic function scores of treated and untreated patients, from the time of admission to the hospital to weeks 1, 2, and 3 are shown in FIG. 3. Only 9 of 17 untreated patients were seen at follow-up visits at the end of weeks 2 and 3.

FIG. 3 s a graphic illustration of combined neurologic function scores, over time, for 15 patients with St. Louis meningoencephalitis treated with interferon-2b and for 17 untreated patients. The numbers in parentheses denote the number of patients seen for follow-up at each weekly interval. Numbers in parentheses denote the number of patients seen for follow-up at each weekly interval.

Quadriparesis or quadriplegia occurred in 8 untreated patients and in 4 treated patients. Seven untreated patients and 3 treated patients had respiratory insufficiency that required ventilatory assistance. Persistence of these complications after the first week of follow-up occurred among 11 untreated patients and 2 treated patients, and persistence after the second week of follow-up occurred among 5 untreated patients and 1 treated patient (table 4).

TABLE 4

| | Patients with severe neurologic impairment or respiratory insufficiency, | | | | | | | | | |
|---|---|---|---|---|---|---|---|---|---|---|
| | by week of hospitalization | | | | | | | | | |
| | Treated with IFN-α2b (n = 15) | | | | | Untreated (n = 17) | | | | |
| Clinical | Week | Week | Week | Week | Week | Week | Week | Week | Week | Week |
| complication^{a} | 0^{b} | 1 | 2 | 3 | 4 | 0^{b} | 1 | 2 | 3 | 4 |
| Quadriplegia | 0 | 0 | 0 | 0 | 0 | 0 | 0 | 2 | 0 | 1 |
| Quadriparesis | 0 | 2 | 1 | 1 | 0 | 0 | 0 | 2 | 2 | 1 |
| Respiratory | 1 | 2 | 0 | 0 | 0 | 0 | 4 | 2 | 0 | 1 |
| insufficiency | | | | | | | | | | |

NOTE.
Data are no. of patients with ≧1 of each of the 3 complications at each weekly follow-up visit; data are not cumulative.
^{a}Of the treated patients, 1 had ≧1 of the 3 clinical complications after 2 weeks of follow-up, and 2 had ≧1 of the 3 clinical complications after 1 week of follow-up. Of the untreated patients, 5 had ≧1 of the 3 clinical complications after 2 weeks of follow-up, and 11 had ≧1 of the 3 clinical complications after 1 week of follow-up.
^{b}Time of admission to the hospital.

Table 4 shows the proportion of patients treated with interferon (IFN)-α2b and untreated patients with severe neurologic impairment or respiratory insufficiency at admission to the hospital and after 1-4 weeks of hospitalization.

All patients, whether treated or untreated, survived after the 4-week follow-up period. The mean length of acute-care hospitalization was 15.0 days for 17 untreated patients and 11.3 days for 15 treated patients.

Eleven of 15 treated patients developed a total WBC count of <4000 WBCs/mm3. Absolute neutrophil counts decreased to <1000 neutrophils/mm3 in 3 patients (all 3 had counts of 900-1 000 neutrophils/mm3), and the counts recovered to >1000 neutrophils/mm3 after IFN-α2b therapy was withheld temporarily. No secondary bacterial infections occurred. Elevation of liver enzyme levels was noted in 3 of 32 patients, untreated and treated, at admission to the hospital. Of the individuals who were treated with IFN-α2b, 5 of 15 developed elevations of liver enzyme levels of 50-200 U/mL during the first week, and 11 of 15 developed elevations of 50-250 U/mL during the second week. Such elevations of liver enzyme levels occurred in 3 of 8 untreated patients, all of whom had peak levels of <200 U/mL. No episodes of severe hepatitis occurred, and all abnormalities resolved after completion of IFN-α2b therapy.

In duplicate experiments, IFN-α2b, in 2-fold increasing concentrations, was added to baby hamster kidney cells 1-2 h after infection with SLE virus. At an IFN-α2b concentration of 23.4 U/mL, an increase in viability of 13%-29% occurred in such cells, compared with infected control cells. Cell viability increased to 49%-68% after the addition of 750 U of IFN-α2b/mL. The addition of IFN-α2b before infection of cells by SLE virus resulted in a greater effect: IFN-α2b concentrations of 23.4-750 U/mL yielded increased cell viability, from 50%-56% to 64%-94%.

Discussion

The results of the present nonrandomized, unblinded, interventional pilot study of the use of IFN-α2b therapy for St. Louis viral meningoencephalitis suggest a potential beneficial effect of IFN-α2b on the early neurologic course of treated patients. However, the absence of randomization in this initial trial introduces the potential for bias and prohibits any definite conclusion regarding the efficacy of IFN-α2b in the treatment of St. Louis viral meningoencephalitis. We consider the results of the present study to be supportive of subsequent prospective, randomized, double-blinded, placebo-controlled trials of IFN-α2b therapy for flavivirus meningoencephalitis. These results do not support current, empirical use of IFN-α2b for the treatment of undiagnosed or suspected flavivirus meningoencephalitis. Such therapy remains experimental pending the results of future controlled trials. St. Louis and West Nile viral meningoencephalitis present with very similar clinical findings and provoke cross-reactive antibody responses. Thus, the diagnostic distinction between these infections may require special studies, such as the plaque reduction neutralization test. The plaque reduction neutralization test confirmed the diagnosis of infection with SLE virus for 4 of the treated patients in the present study, as well as for 34 of 70 case patients from the same geographic area whose cases were reported by the Louisiana Office of Public Health. Entomological studies performed by the CDC demonstrated the prevalence of infection with SLE virus to be 3-5 cases/1000 C. quinquefasciatus mosquitoes. These data provide strong supportive evidence for SLE virus as the cause of infection among our treated and untreated patients.

The results of the present study are consistent with findings regarding the therapeutic effect of IFN-α2b against flavivirus encephalitis in an animal model. A study of the use of IFN-α2b for the treatment of flavivirus (Modoc virus) encephalitis in SCID mice showed a significantly increased mean survival time for treated mice, compared with untreated mice. Treatment significantly reduced the levels of viral RNA in the serum, brain, and spleen.

The present study follows the suggestion made by Merigan [p. 2515], in 1982, that the action of IFN-α2b against “CNS viral infections that are particularly severe and caused by RNA viruses for which we have no other available therapy” be examined. The present study was initiated as an emergency intervention, in an attempt to lessen the neurologic effects associated with an ongoing outbreak of severe SLE viral meningoencephalitis. Thus, a placebo-controlled, randomized, blinded study was not possible. However, the proportion of patients who experienced quadriplegia, quadriparesis, or respiratory insufficiency after the first or second week of hospitalization was greater among untreated patients than among patients who received IFN-α2b. The mean neurologic function score at admission to the hospital was similar among treated and untreated patients.

The present study has demonstrated that IFN-α2b is active against SLE virus in vitro; that 2 weeks of treatment with IFN-α2b for CNS infection due to SLE virus is well tolerated, except for the development of transient neutropenia and/or mild hepatitis; and that the clinical course of treated patients appears to be favorable, compared with that of untreated patients, within the same outbreak at the same hospital. A definitive conclusion regarding the beneficial effect of IFN-2b against St. Louis or other flavivirus meningoencephalitis awaits a placebo-controlled, prospective, randomized, blinded study. On the basis of the results of this pilot study, we conclude that further investigation of early initiation of IFN-α2b therapy for previously untreatable CNS flavivirus infection is warranted.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed:

1. A method of treating a human suffering from a meningitis, encephalitis, meningo-encephalitis or encephalomyelitis caused by an arbovirus, comprising parenterally administering to the human an effective amount of interferon.

2. A method according to claim 1, wherein the interferon comprises interferon-alpha.

3. A method according to claim 2, wherein the interferon-alpha is administered intravenously, intramuscularly, or subcutaneously or combination thereof.

4. A method according to claim 2, wherein the interferon alpha comprises interferon alpha-2a, interferon alpha-2b, a consensus interferon, pegylated interferon-alpha-2a, pegylated interferon-alpha-2b, pegylated consensus interferon or combination thereof or a combination of interferon alpha species.

5. A method according to claim 2, wherein the human is suffering from West Nile virus, Japanese encephalitis virus, St. Louis encephalitis virus or Murray Valley encephalitis virus, eastern equine encephalitis, western equine encephalitis, LaCrosse encephalitis virus, tick-borne encephalitis, Venezuelan equine encephalitis or Powassan virus encephalitis.

6. A method in accordance with claim 4, wherein the interferon is alpha-2b or alpha-2a interferon and is administered in an amount from about 1.5 million units to about 10 million units/day.

7. A method in accordance with claim 4, wherein the interferon alpha-2a or alpha-2b is administered in an amount of 3 million units as an initial dose intravenously, then 3 million units every 12 to 24 hours subcutaneously.

8. A method of preventing or treating a human suffering from a meningitis, encephalitis, meningo-encephalitis or encephalomyelitis caused by a West Nile virus, comprising parenterally administering to the human an effective amount of interferon.

9. A method according to claim 8, wherein the interferon alpha comprises interferon alpha-2a, interferon alpha-2b, a consensus interferon, pegylated interferon-alpha-2a, pegylated interferon-alpha-2b, pegylated consensus interferon or combination thereof or a combination of interferon alpha species.

10. A method in according to claim 8, wherein the interferon is administered with ribavirn.

11. A method in accordance with claim 10, wherein the ribavirin is administered in an amount from about 300 mg to about 3600 mg/day.

12. A method of treating a human suffering from a meningitis, encephalitis, meningo-encephalitis or encephalomyelitis caused by St. Louis encephalitis virus, comprising parenterally administering to the human an effective amount of interferon.

13. A method according to claim 12, wherein the interferon comprises interferon-alpha.

14. A method according to claim 13, wherein the interferon-alpha is administered intravenously, intramuscularly, or subcutaneously or combination thereof.

15. A method according to claim 13, wherein the interferon alpha comprises interferon alpha-2a, interferon alpha-2b, a consensus interferon, pegylated interferon-alpha-2a, pegylated interferon-alpha-2b, pegylated consensus interferon or combination thereof or a combination of interferon alpha species.

16. A method in accordance with claim 15, wherein the interferon alpha-2a or alpha-2b is administered in an amount of 3 million units as an initial dose intravenously, then 3 million units every 12 to 24 hours subcutaneously.

17. A method of treating a human suffering from a meningitis, encephalitis, meningo-encephalitis or encephalomyelitis caused by a West Nile virus, comprising intravenously and/or subcutaneously administering to the human an effective amount of interferon-alpha.

18. A method according to claim 17, wherein the interferon alpha comprises interferon alpha-2a, interferon alpha-2b, a consensus interferon, or a combination of interferon alpha species.

19. A method in accordance with claim 18, wherein the interferon alpha-2a or alpha-2b is administered in an amount of 3 million units as an initial dose intravenously, then 3 million units every 12 to 24 hours subcutaneously.
